(19) United States
(12) Patent Application Publication
(10) Pub. No.: US 2025/0182521 A1
(43) Pub. Date: Jun. 5, 2025

(54) SENSORS ASSEMBLY WITH AN OPTICAL UNIT DECENTERED IN AN ELECTRODE

(71) Applicant: Withings, ISSY LES MOULINEAUX (FR)

(72) Inventors: Jorys TINÉ, ISSY LES MOULINEAUX (FR); Thomas BEDETTI, ISSY LES MOULINEAUX (FR)

(21) Appl. No.: 18/969,683

(22) Filed: Dec. 5, 2024

(30) Foreign Application Priority Data
Dec. 5, 2023 (FR) 2313600

(51) Int. Cl. G06V 40/13

(52) U.S. Cl. CPC G06V 40/1318; G06V 40/1324

(57) ABSTRACT

A sensors assembly for physiological measurements includes an electrode including a contact surface, the contact surface being configured to be in contact with a user, the contact surface being elongated in a main direction, an optical unit including an optical sensor and an interaction surface configured to be in contact with a user. The contact surface includes a first section and a second section along the main direction. The interaction surface is positioned along the main direction between the first section and the second section. The first section is longer than the second section along the main direction.

### CROSS-REFERENCE TO RELATED APPLICATIONS

This application claims priority to French Patent Application No. FR2313600, filed Dec. 5, 2023, the entire content of which is incorporated herein by reference in its entirety.

### FIELD

The present description relates to a sensors assembly for physiological measurements.

The present description also relates to portable physiological measurement devices (hereinafter the measurement device), for example of the “personal hand-held monitor” (PHHM) type, incorporating such a sensors assembly for physiological measurement. Such devices may be used for remote monitoring, for example by a doctor during a teleconsultation or asynchronous consultation.

### BACKGROUND

Numerous devices for measuring electrocardiogram (ECG) and/or PhotoPlethysmoGraphy (PPG) are known. Sensors may be integrated within the same device, as on a connected watch for example. Examples include the Withings ScanWatch™, the Apple Watch™ and the Samsung Galaxy™. These watches are configured to measure ECG and PPG separately.

It has also been proposed to measure ECG and PPG simultaneously with the same device. Examples include documents U.S. Ser. No. 10/709,339B1, US20220175321A1, US20210076957A1.

Nevertheless, these devices present a number of limitations, notably in terms of measurement quality and ergonomics, particularly with a view to obtaining devices that may be easily produced and distributed on a large scale, rather than just prototypes.

### SUMMARY

The description relates to sensor assemblies for physiological measurements enabling simultaneous ECG and PPG measurement with improved quality and ergonomics.

To this end, an aspect of the present description relates to a sensors assembly for physiological measurements comprising:
an electrode comprising a contact surface, the contact surface being configured to be in contact with a user, the contact surface being elongated along a main direction,
an optical unit comprising an optical sensor and an interaction surface configured to be in contact with a user,
wherein the contact surface comprises a first section and a second section along the main direction,
wherein the interaction surface is positioned along the main direction between the first section and the second section,
and wherein the first section has a greater length than the second section along the main direction.

In an embodiment, the contact surface defines an aperture, with the interaction surface positioned in the aperture.

In an embodiment, the electrode extends around the opening for at least 1 mm.

In an embodiment, the opening is off-center from the contact surface along the extension direction.

In an embodiment, the contact surface surrounds the interaction surface.

In an embodiment, the first and second sections of the electrode contact surface are separated.

In an embodiment, the contact surface is adjacent to the interaction surface of the optical unit.

In an embodiment, adjacent means that no mechanical parts are arranged between the contact surface and the optical unit.

In an embodiment, the contact surface is separated from the optical unit by a gap of less than 3 mm, in particular less than 1 mm.

In an embodiment, the center of the interaction surface is located between 55% and 95%, or even between 65% and 85%, of a length of the contact surface in the main direction.

In an embodiment, the contact surface has a convex shape.

In an embodiment, the contact surface extends as an extension of the housing.

In an embodiment, the contact surface has a concave shape.

In an embodiment, the contact surface forms a trough.

In an embodiment, the contact surface extends over a length of between 1 cm and 5 cm in the main direction.

In an embodiment, the contact surface has an oblong or rectangular shape.

In an embodiment, the contact surface is metallic.

In an embodiment, the electrode is an ECG electrode.

In an embodiment, the electrode is an impedance measurement electrode, IPG.

In an embodiment, the optical sensor comprises a light source and a light receiver, the light source and light receiver being aligned in a direction orthogonal to the main direction in which the contact surface extends.

In an embodiment, the optical unit comprises a dome, the dome comprising the interaction surface, the interaction surface extending in a plane parallel to a plane tangent to the contact surface.

In an embodiment, at least part of the dome has a cylindrical or parallelepiped shape.

In an embodiment, the interaction surface is flat.

In an embodiment, the interaction surface has a maximum transverse dimension of between 3 mm and 10 mm.

In an embodiment, the interaction surface has an area twice, beneficially three times, smaller than the area of the contact surface.

The present description also relates to a device comprising a housing and the sensors assembly as defined above, the sensors assembly being arranged on the housing.

In an embodiment, the electrode is mounted on the housing and the contact surface has a concave shape relative to the housing so as to form a trough for receiving the finger.

In an embodiment, the electrode has a curved shape towards the inside of the housing.

In an embodiment, the contact surface forms a recess in the housing.

In an embodiment, the housing has an elongated shape along a direction of extension between two ends, the main direction of the contact surface being parallel to the direction of extension of the housing.

In an embodiment, the contact surface is located near one end of the device along the direction of extension, the first section being located between the end and the second section along the direction of extension.

In an embodiment, the contact surface near the end of the device along the extension direction is disposed between a first edge of the housing and strictly a quarter of a length of the housing along the extension direction from the first edge.

In an embodiment, a section of the housing is oblong or rectangular, wherein the housing comprises a front face, a rear face, a top face, and a bottom face, opposite the top face, wherein the front and rear faces have a height greater than the depth of the bottom and top faces.

In an embodiment, the sensors assembly is positioned on a top face of the housing.

In an embodiment, the device is holdable with two hands in use.

In an embodiment, the device comprises an optical module configured to generate instructions enabling the transmitter to emit light and configured to receive signals from the light receiver, notably to determine a heart rate or oxygen saturation of the user's blood.

In an embodiment, the device comprises a second ECG electrode.

In an embodiment, the device comprises an ECG module connected to the two ECG electrodes and configured to measure an ECG signal.

In an embodiment, the device comprises a control unit configured to control the simultaneous measurement of an ECG and an optical measurement.

In an embodiment, the device comprises a second IPG electrode.

In an embodiment, the device comprises an IPG module connected to the two IPG electrodes and configured to perform an impedancemetry measurement.

In an embodiment, the device comprises a second IPG electrode.

In an embodiment, the device comprises an IPG module connected to the two IPG electrodes and configured to perform an impedancemetry measurement.

In an embodiment, the device comprises a wave module configured to calculate a propagation velocity of a pulse wave in a user's arm based on signals received from the ECG module and the optical module.

The present description also relates to a physiological measurement sensors assembly comprising: an electrode comprising a contact surface, the contact surface being configured to be in contact with a user, the contact surface being elongate along a direction of extension, the contact surface defining an aperture, the aperture being off-center from the contact surface along the direction of extension, an optical sensor arranged in the opening.

In an embodiment, off-center means that the barycenter of the contact surface and the barycenter of the opening do not coincide.

In an embodiment, the contact surface is located near one end of the device along the direction of extension, the opening being arranged in the contact surface on the side opposite the end.

### BRIEF DESCRIPTION OF THE DRAWINGS

Further features, details and benefits will become apparent from the detailed description below, and from an analysis of the appended drawings, on which:

FIG. 1 This figure shows two perspective views, right and left, of a measuring device comprising a set of sensors according to one embodiment.

FIG. 2 This figure shows four projected views of the device shown in FIG. 1.

FIG. 3 This figure shows a view of the device in two-handed operation.

FIG. 4 This figure shows three top views (a), (b), (c) of the sensors assembly in three different variants.

FIG. 5 This figure shows a top view (a) and a vertical section (b) of the sensors assembly integrated into the device.

FIG. 6 This figure shows two perspective views (a), (b) of the sensors assembly.

FIG. 7 This figure shows two side views (a), (b) of the sensors assembly in the rest position and in the displaced position.

FIG. 8 This figure shows a vertical cross-sectional view of the device, with schematic components.

FIG. 9 This figure shows a perspective view and a cross-sectional view of an optical unit in the sensors assembly.

FIG. 10 This figure shows two perspective views of a transparent part of the optical unit shown in FIG. 9.

FIG. 11 This figure shows two projected views, front and rear, of a measuring device in another embodiment.

FIG. 12 This figure shows a top view and vertical section of the sensors assembly integrated into the device shown in FIG. 11.

FIG. 13 This figure shows a schematic view of the device with certain components, particularly electronic ones.

### DETAILED DESCRIPTION

### Overview of the Device

The present description will describe several embodiments and variants of sensor assemblies, in particular integrated into physiological measurement devices. The term “device” will be used to simplify the language.

The device integrates the sensor array and optionally one or more other physiological sensors to measure physiological characteristics (“physiological measurement”) of a user who is also the manipulator.

Physiological measurements are measurements of a user's physiological characteristics (hereinafter referred to as “user”) that reflect a health status, such as temperature, heart sounds, lung sounds, heart rate, arrhythmia, etc.

The device may be portable, i.e. lightweight and space-saving. This portability allows users to easily hold it in one hand and conveniently store it in locations such as a drawer, handbag, or pocket. For example, the device weighs less than 250 g, or even 150 g. Its dimensions are also compact with a volume of less than 20×10×10 cm, or even 20×5×5 cm, or even 15×5×5 cm.

In addition, the device may be connected, in the sense that it may send data to a third-party device, such as a smartphone or server. This connectivity enables the device to function as an RPM device, with the user becoming a patient of a remote doctor. Teleconsultation may be synchronous, with live or quasi-direct interaction with the doctor, or asynchronous. In synchronous mode, the patient uses the device to acquire physiological data, which are immediately or almost immediately transmitted to the doctor (a few seconds later). In asynchronous mode, the patient uses the device when he or she can, and the doctor consults the physiological data when he or she can, at a different time if necessary.

The device is designed to be used in particular for self-measurement, ensuring ease of use so that individuals can effortlessly perform measurements on themselves.

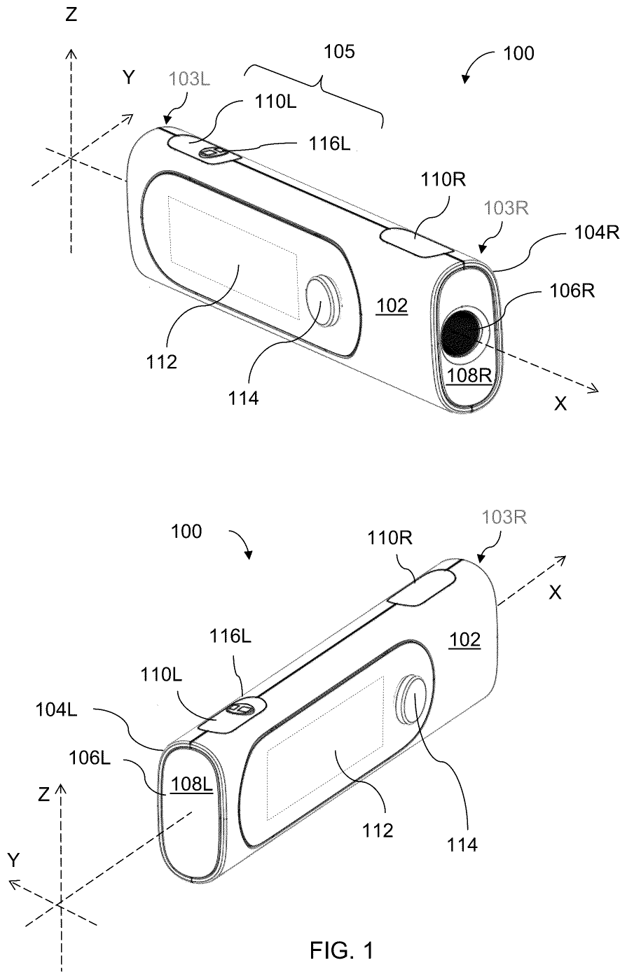

FIGS. 1 to 3 show a device 100 according to an embodiment.

### The Housing

The device 100 comprises an elongated housing 102 that defines an extension direction X. The housing 102 also defines two orthogonal transverse directions Y and Z. Hereafter, the notions of “longitudinal” and “transverse” are defined in relation to the extension direction X. The device 100 has its largest dimension along this extension direction X. The direction of extension X is rectilinear in the figures, but curvature is possible provided that handling of the device is not significantly impaired.

Along the extension direction X, the housing 102 comprises a first end 103L or first distal end (L for “Left”) and a second end 103R or second distal end (R for “Right”), opposite the first end 102L. Each end 103L, 103R may extend along the transverse plane YZ, orthogonal to the extension direction X. The first end 103L defines a first edge 104L and the second end 103R defines a second edge 104R. Each edge 104L, 104R defines a closed curve (ovoid in the figures due to the cross-section of housing 102 at first end 103L and second end 103R).

To enable an easy holding, each edge 104L, 104R is less than 30 cm or even 15 cm long.

In an embodiment, the distance L between the two edges 104L, 104R along the extension direction X is less than 20 cm, or even 15 cm. This distance L ensures that the device 100 is portable.

The housing 102 may be essentially cylindrical in shape, with a convex cross-section in the YZ plane (i.e. orthogonal to the extension direction X). In an embodiment, this cross-section has two axes of symmetry, for example the Y and Z axes as shown in the figures. The cross-section is, for example, oblong, as shown in the figures, or rectangular (with more or less rounded corners), or circular.

Essentially cylindrical means that the section orthogonal to the extension direction X has no significant dimensional variation. For example, “essentially cylindrical” refers to a shape whose cross-section, taken along a plane perpendicular to the extension direction X, exhibits minimal or no significant variation in dimensions across its length. In practical terms, this implies that the width and height of the cross-sectional shape remain substantially uniform, resulting in a consistent, cylindrical appearance. Minor deviations or surface features that do not significantly alter the overall uniformity of the dimensions can still fall within this definition. This characteristic ensures structural simplicity, aesthetic consistency, and ease of handling, which are relevant for the device's ergonomic and functional design.

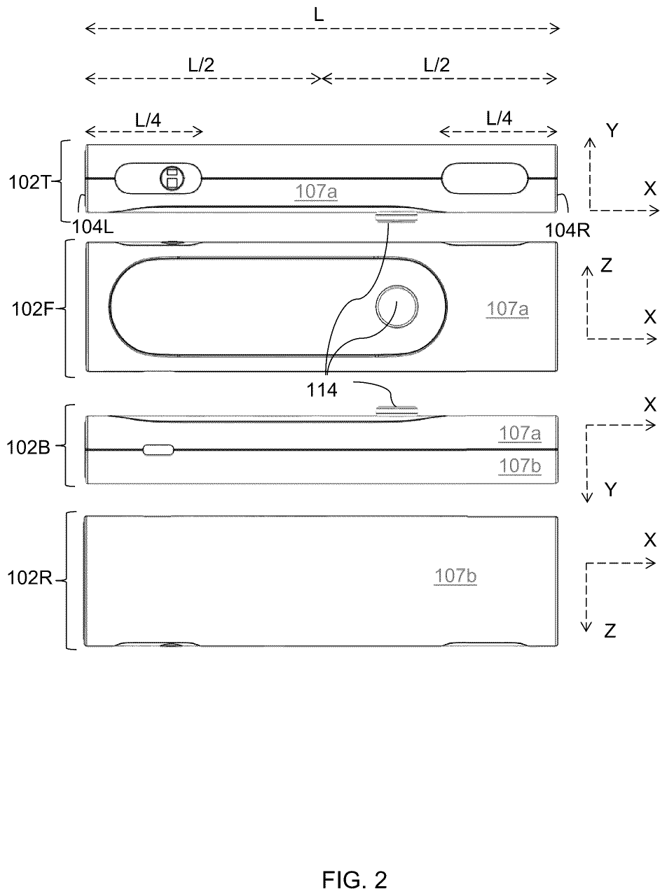

The housing 102 is typically made of plastic, to be lightweight, cost-effective and electrically insulating. When the user holds the device 100, his or her hand(s) are mostly in contact with the housing 102. The housing 102 may be made up of several parts assembled together. In FIGS. 1 and 2, the housing 102 comprises two shells 107, noted respectively 107a, 107b, which may be assembled at a junction parallel to the extension direction X. Alternatively, the housing 102 is formed from a single shell. Other types of assembly are also possible.

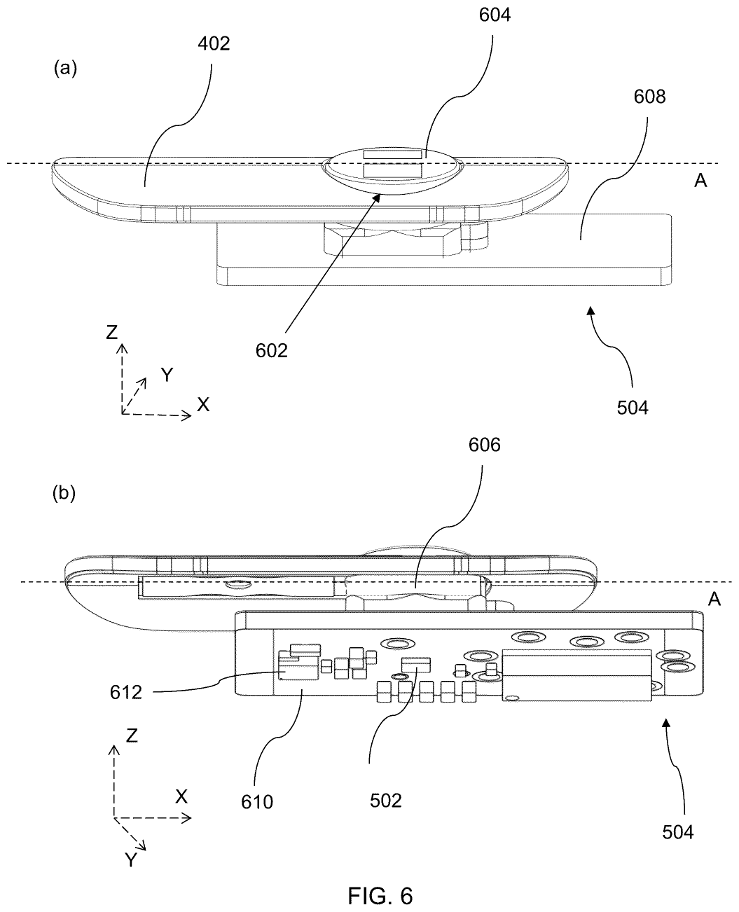

At least two faces connecting the two edges 104L, 104R may be defined for the housing 102. In the case of an oblong or rectangular cross-section, a front face 102F (F for “Front”), a rear face 102R (R for “Rear”) (opposite to the front face), a top face 102T (T for “Top”), and a bottom face 102B (B for “Bottom”), opposite to the top face, are defined, as illustrated in FIG. 2; finally, the two edges 104L, 104R define two lateral surfaces. These face names are defined in relation to the two-handed handling position illustrated in FIG. 6.

As shown in FIG. 2, each of the top face 102T and bottom face 102B extends mainly in a longitudinal plane XY. Each of the front face 102F and rear face 102R extends mainly in a longitudinal plane XZ, orthogonal to the XY plane. Each of the two edges 104L, 104R extends mainly in a transverse plane XZ, i.e. the side faces are also orthogonal to the extension direction X. Rounded shapes for the front face 102F, rear face 102R, top face 102T and bottom face 102U may be provided, as illustrated in the figures, to have no edge or thus facilitate holding.

The front face 102F and rear face 102R have a height (the Z dimension) greater than the depth (the Y dimension) of the bottom face 102B and top face 102T. In other words, the housing 102 is taller than it is deep.

The front face 102F and the rear face 102R have a longer length along the X dimension than they are tall in the Z dimension. In other words, the housing 102 is designed to be longer than it is tall.

These size considerations similarly apply to device 100. Device 100 has a length along the X dimension that is greater than its height Z dimension which, in turn, is itself greater than its depth (Y dimension). For example, the length is three (3) times greater than the height and the height is 1.5 times greater than the depth. These dimensions align with the specified volumes for the device represented as X-dimension, Y-dimension, and Z-dimension.

The device 100 includes a set of sensors 105, which will be described in more detail later.

The device 100 may also comprise one or more additional physiological sensors 106 located at different locations on the housing 102, enabling additional physiological measurements to be taken and physiological data to be generated.

### The Display

The device 100 comprises a display 112, for example a screen (shown dotted in FIG. 1 because the screen contour is invisible to the user, or at least only slightly visible, in this embodiment), for displaying information and/or measurement results to the user. In an embodiment, the display 112 is configured to display information along a reading direction parallel and/or transverse to extension direction X. In an embodiment, the device 100 comprises a gyrometer configured to determine the orientation in space of the device 100 and adapt the reading direction as a function of this orientation. In this way, the display 112 may be transverse when the user holds the device with one hand and longitudinal when the user holds the device with two hands. In an embodiment, the display 112 is positioned on the front face 102F of the housing 102, so that the user may easily see the display 112 when he/she manipulates the device.

### The Physical Interface

The device 100 also includes a physical interface 114 with the user, which may take the form of a joystick, arrow, etc. The physical interface 114 is functionally or operably connected to the display 112 and enables navigation through a menu displayed on the display 112. The physical interface 114 is functionally connected to the display 112 and enables navigation through a menu displayed on the display 112. The physical interface 114 may be positioned on the front face 102F of the housing 102.

To simplify navigation, the display 112 and the physical interface 114 are positioned side by side, for example on the front face 102F. In the embodiment shown in FIG. 1, the physical interface 114 is positioned on the side of the second end 103R.

### The Sensor Assembly

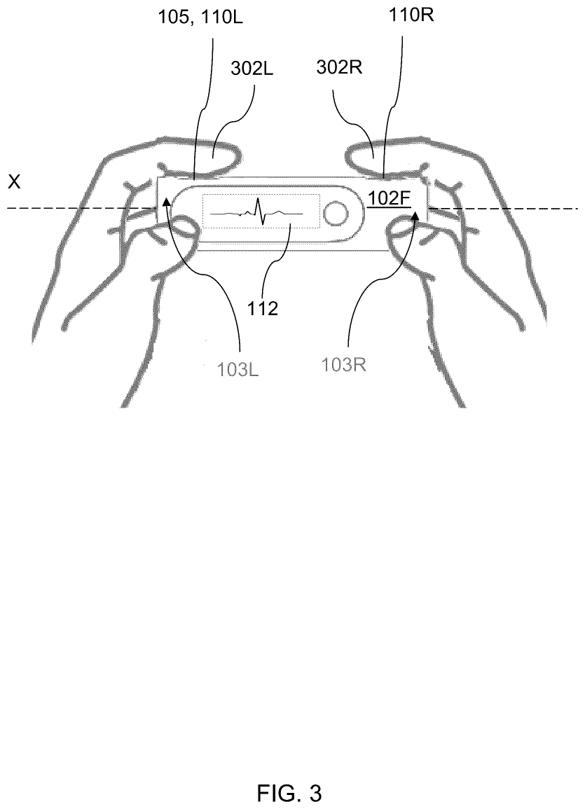

As previously mentioned, the device 100 comprises at least one sensors assembly 105. The sensors assembly 105 is designed to receive a user's finger (index finger or thumb, for example). In particular, as illustrated in FIG. 3, the sensors assembly 105 is positioned under a finger, in particular the index finger 302, in the two-handed gripping position of the device 100.

The sensors assembly 105 is positioned on the housing 102, for example, and may be arranged at various locations on the housing 102. In particular, the sensors assembly 105 is located near an end, in particular the first end 103L. According to one embodiment, “near an end” means “between the edge of the end and strictly half the length L from the edge”. According to another embodiment, “near” means “between the first edge 104L and strictly a quarter of the length L from the first edge 104L”. These examples may be seen in FIG. 2, with distances L/2 and L/4 shown.

As illustrated in FIGS. 1 to 3, the sensors assembly 105 may be positioned so that the user's index finger 302L, 302R is naturally positioned on it. In this respect, the sensors assembly 105 may be positioned on top face 102T.

In a non-illustrated embodiment, the sensors assembly 105 falls below the thumb. In this respect, the sensors assembly 105 is positioned on the front face 102F.

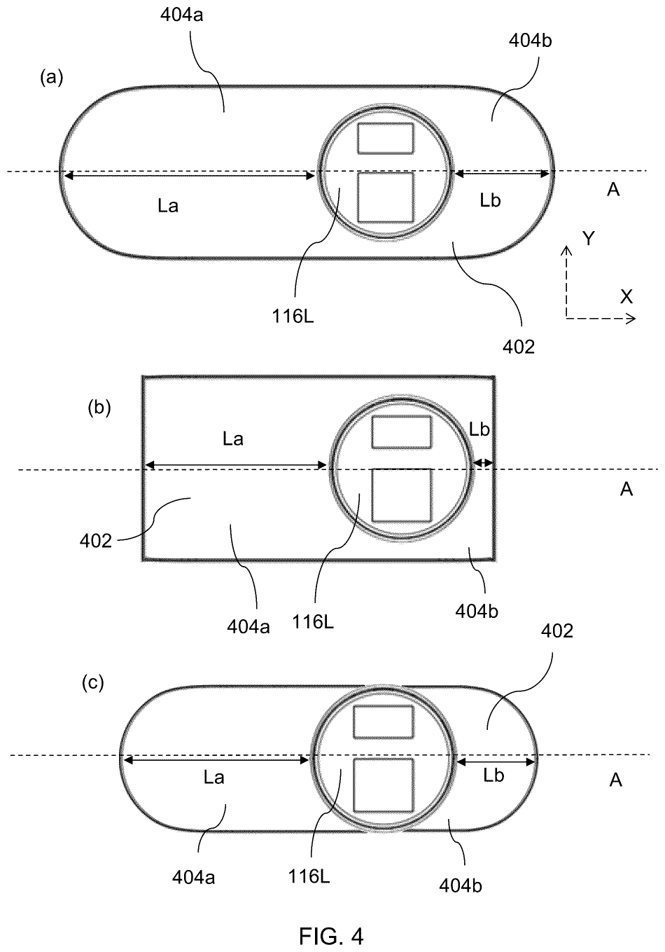

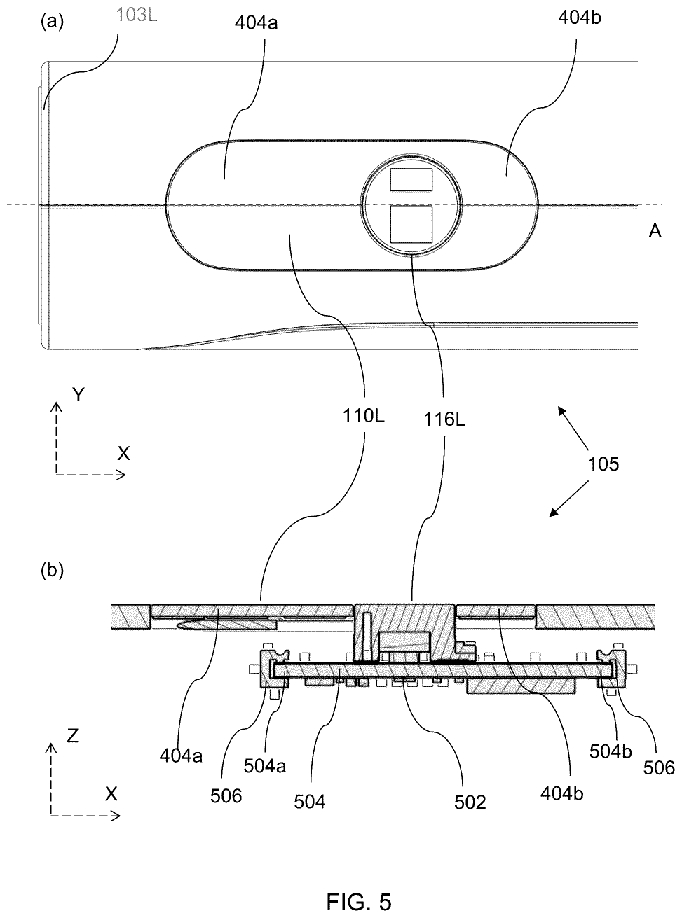

Referring to FIGS. 4 and 5, the sensors assembly 105 comprises an electrode 110L, hereinafter referred to as the first electrode, an optical unit 116L and a force sensor 502.

### The First Electrode

The first electrode is a conductive sensor that conducts an electric current from the user's body or injects it into the user's body.

In an embodiment, the first electrode is an electrocardiogram electrode, referred to as the first ECG electrode 110L.

Alternatively, the first electrode 110L is an impedance measurement electrode, known as the first IPG electrode 110L, in particular for determining the user's body composition (muscle mass, fat mass, etc.).

Alternatively, the first electrode 110L is a combined ECG and IPG electrode. In this case, electrode 110L is connected to a connector that enables switching from one measurement to the other.

The first electrode 110L may be fixed with respect to the housing 102.

The first electrode 110L is made of conductive material, such as metal (e.g. stainless steel or titanium alloy). In the example shown in the figures, the first electrode 110L is in the form of a metal body. By metal body we mean a part whose maximum thickness is greater than 0.1 mm. In one variant, the first electrode 110L may be formed by a conductive coating deposited on a surface (which is itself conductive or non-conductive).

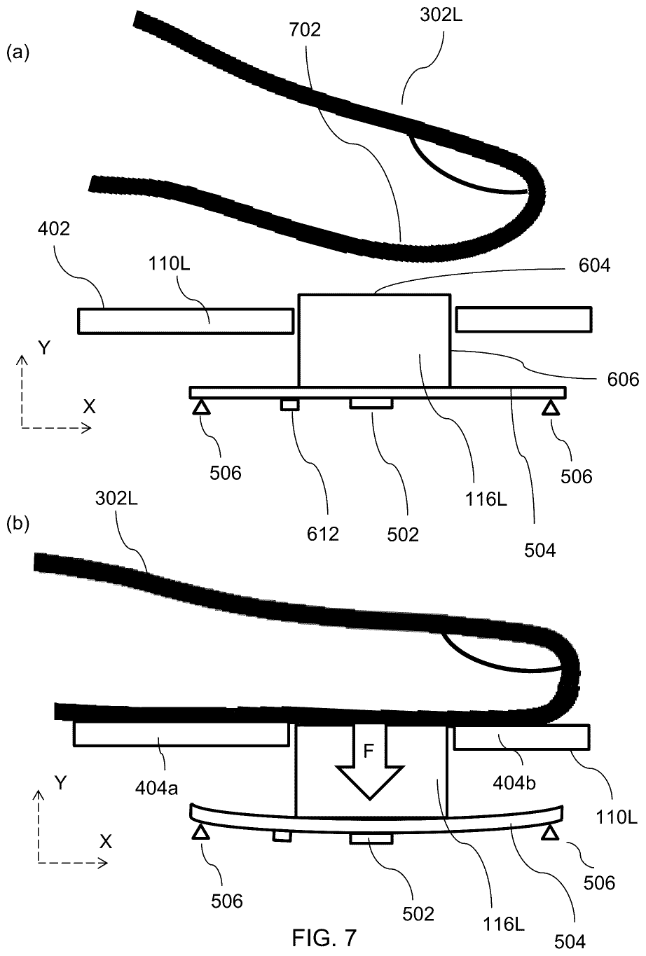

With reference to FIGS. 4 to 7, the first electrode 110L comprises a contact surface 402. The contact surface 402 is configured to be in contact with a user, in particular the user's finger as illustrated in FIG. 7.

As shown in FIG. 6, the contact surface 402 may have a convex shape. In other words, the contact surface 402 has a convex shape towards the outside of the housing 102. In particular, the contact surface 402 extends in a flush manner or aligns seamlessly with the housing 102. In this way, there is no surface discontinuity between the housing 102 and the electrode 110L. This design enhances comfort by ensuring a pleasant grip while facilitating effortless contact between the electrode and various parts of the body, possibly other than the finger, such as the torso, for example.

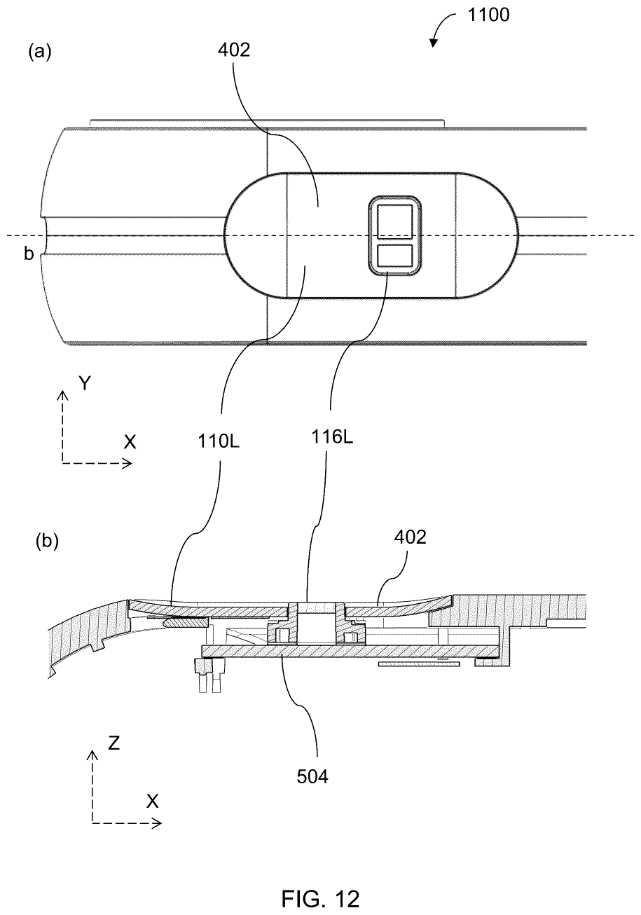

Alternatively, as shown in FIG. 12, the contact surface 402 may have a concave shape relative to the housing 102. In other words, the contact surface 402 has a curved shape towards the inside of the housing 102, or forms a recess in relation to the housing. In this way, the contact surface 402 forms a trough suitable for receiving a finger, in particular, to better match its shape and stabilize it during measurement. The contact surface 402 guides the user's finger over the electrode, enabling stable, easy measurement for the user. In a variant not shown, the concave shape extends (e.g. with the same width) over the housing 102 between the electrode 110L and the edge 104L, to further improve finger guidance.

In a variant not shown, the contact surface 402 has a substantially flat shape. The term “substantially” in this context indicates that the surface is predominantly flat, with any deviations being minor and not significantly affecting the overall flatness. These deviations might include slight curvature, surface texture, or other small variations that do not compromise the primary functionality or appearance of the surface as flat. This design ensures the contact surface remains practical for maintaining consistent contact with the user while potentially accommodating ergonomic or aesthetic enhancements.

According to the embodiments shown in FIG. 4, the contact surface 402 is elongated. The contact surface 402 extends along a main direction A. The main direction A may be parallel to the direction of extension X of the housing 102. In this way, the electrode 110L extends in the same direction as the housing 102. The contact surface 402 may have an oblong shape, as shown in views (a) and (c) of FIG. 4. Alternatively, the contact surface 402 may have a rectangular shape, as shown in view (b) of FIG. 4. Other shapes are also possible. The contact surface 402 may extend over a length of between 1 cm and 5 cm in the main direction A. The contact surface 402 may extend over a width (the dimension along the Y direction) of between 5 mm and 12 mm (even when the contact surface 402 is slightly convex).

### The Optical Unit

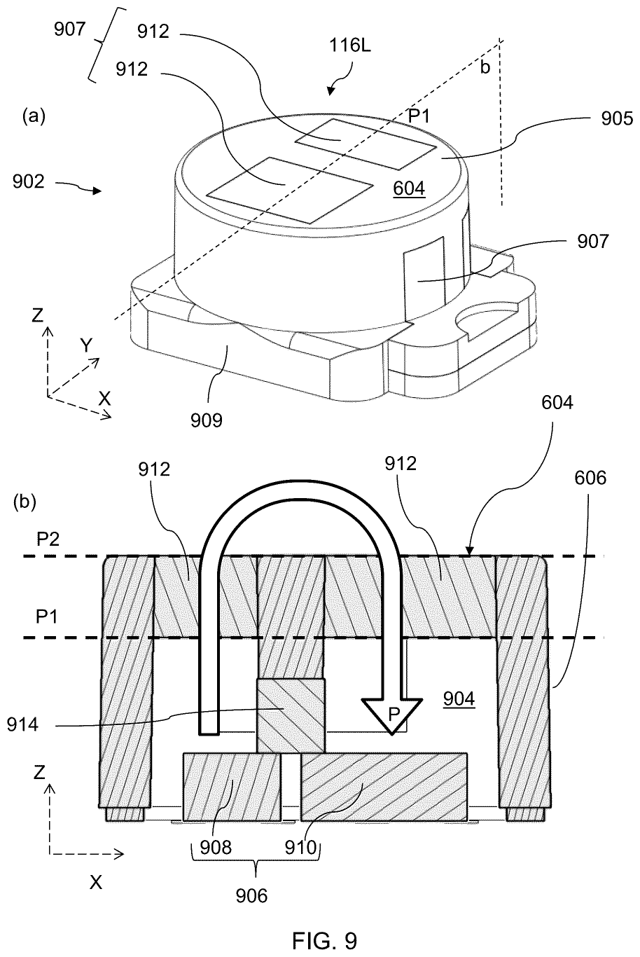

Referring to FIG. 9, the optical unit 116L comprises an interaction surface 604, configured to be in contact with a user (e.g. the index finger), and an optical sensor 906. In an embodiment, the optical unit 116L comprises a dome 902 defining an interior volume 904 within which optical sensor 906 is arranged. The dome 902 also comprises the interaction surface 604. The structure of dome 902 will be described in more detail later.

The optical sensor 906 comprises at least one light source 908 and a light receiver 910. The light source 908 is configured to emit light and the light receiver 910 is configured to receive light from the light source 908, in particular light that has passed through the user, for example his index finger 302L.

In an embodiment, the light source 908 and the light receiver 910 are aligned in a direction orthogonal to the main direction A along which the contact surface 402 extends.

The optical sensor (in particular the light source 908 and light receiver 910) may be mounted on a carrier 504. The carrier 504 may be a printed circuit board (PCB), hereinafter referred to as an optical printed circuit board or optical PCB. In the latter case, the optical sensor is electronically connected to the optical PCB.

The bracket 504 is attached to housing 102. The bracket 504 will be described in more detail later.

The light source 908 may comprise one or more LEDs, such as a green LED, a red LED and at least one infrared LED. The light receiver 910 may be a photoreceiver, such as a photodiode. The light source 908 may comprise a laser.

The device 100 may include an optical module 612. Typically, the optical module 612 comprises an analog-to-digital converter (ADC) and a processor. The optical module 612 may be mounted on the rear side of the optical printed circuit board 504. The optical module 612 is configured to generate instructions for the light source 908 to emit light and configured to receive signals from the receiver 910. The optical module 612 may be configured to determine the user's heart rate and/or blood oxygen saturation on the basis of the optical signals received.

The dome 902 comprises the interaction surface 604 and a side surface 606. The interaction surface 604 is configured to be in contact with a user, in particular the user's finger. the side surface 606 is configured to face the thickness of electrode 110L. The side surface 606 and part of the interaction surface 604 may be one-piece. The dome 902 may further comprise a base 909, also integral with side surface 606.

As shown in FIG. 9, the light source 908 is configured to emit light out of the dome 902 through the interaction surface 604, and the light receiver 910 is configured to receive light from outside the dome 902 through the interaction surface 604. In this way, a main optical path P is defined for light between the light source 908 and the light receiver 910, passing outside the dome 902.

The interaction surface 604 may be flat. In particular, the interaction surface 406 extends in a plane XY parallel to a plane tangent to the contact surface 604. However, the interaction surface 604 may be slightly convex.

The interaction surface 604 may have a maximum transverse dimension (in the Z direction) of between 3 mm and 10 mm. In particular, the interaction surface 604 may be round, but other shapes are envisioned in other embodiments. The maximum transverse dimension is then the diameter.

In an embodiment, the interaction surface 604 has an area twice, beneficially three times, smaller than the area of the contact surface 402.

### Longitudinal Off-Centering of the Optic in the Electrode

According to the embodiments shown in FIG. 4, the contact surface 402 comprises at least two sections 404, including a first section 404a and a second section 404b. The two sections 404a, 404b are electrically connected to form electrode 110L. the electrode 110L then forms a single electrode. The first section 404a and the second section 404b are arranged along the main direction A. The interaction surface 604 of the optical unit 116L is positioned along the main direction A between the first section 404a and the second section 404b. The first section 404a may have a length La greater than a length Lb of the second section 404b along the main direction A. In an embodiment, the length La of the first section 404a is at least 50% greater, beneficially 100% greater, than the length Lb of the second section 404b. This is referred to as longitudinal off-centering of the optical unit 116L within the contact surface 402. The choice of length and its proportional difference between the two sections is relevant for ensuring proper electrode coverage during use. A longer first section 404a accommodates the larger, proximal part of the finger, allowing for a stable and reliable contact area, which is relevant for accurate electrical signal capture. The second section 404b, being shorter, provides complementary coverage for the distal part of the finger without compromising comfort or usability. The range of length ratios is carefully selected to balance ergonomic factors and ensure that the electrode captures consistent and high-quality data. Additionally, the off-centering ensures that the optical unit 116L remains unobstructed while maximizing the electrode's contact with the finger, further enhancing measurement reliability.

As shown in FIG. 7, this off-centering of the optical unit 116L enables to maximize contact between the electrode and the user's finger. Indeed, the user places the pad of his index finger on the interaction surface 604 of the optical unit 116L. The proximal part of the index finger is placed on the first section 404a and the distal part of the index finger, smaller than the proximal part, is placed on the second section 404b. In this way, all, or at least most, of the electrode is covered, providing a better electrical signal, particularly ECG, and limiting external disturbances.

As shown in view (c) of FIG. 4, the first section 404a and the second section 404b of the contact surface 402 may be detached (e.g. visually separated). The first section 404a and the second section 404b may be two different pieces, yet electrically connected, for example by an electric wire extending between the two sections 404 inside the housing 102, or the same part whose contact surface comprises the two detached sections 404a, 404b.

Nevertheless, to ensure that the finger is in contact with both the interaction surface 604 of the optical unit 116L and the contact surface 402 of the electrode, the interaction surface 604 of the optical unit 116L has a dimension in the Y direction less than or equal to that of the electrode 110L (i.e. the width of the electrode).

Alternatively, as shown in views (a) and (b) of FIG. 4, the first section 404a and the second section 404b may be joined together. In other words, the two sections 404 form a single piece.

In this variant, the contact surface 402 may surround the interaction surface of the optical unit 116L. The contact surface 402 then defines an opening 602. The opening 602 is longitudinally off-center with respect to the contact surface 402 along the main direction A. By off-center, it is understood that the barycenter of the contact surface 402 and the barycenter of the opening 602 are not coincident. The optical unit 116L, including the interaction surface 604, is arranged in the aperture 602. In an embodiment, the center of the interaction surface 604 is located between 55% and 95%, such as between 65% and 85%, of the length of the contact surface 402 along the main direction A.

The electrode 110L extends around the opening 602 for at least 1 mm, particularly in a direction transverse to main direction A (direction Y). For reasons of ergonomics and contact with the electrode, the opening 602 is transversely centered (same electrode distance on either side along direction Y).

As shown in FIG. 5, the contact surface 402 is located near one end 103L of device 100 along extension direction X. The first section 404a being located between the end 103L and the second section 404b along the extension direction X. In other words, in the variant where the two sections 404 are joined, the opening 603 is arranged in the contact surface 402 on the opposite side to the end 103L.

As may be seen in FIG. 3, when the user operates the device 100, the proximal phalanges of the index fingers 302L, 302R lie in the extension direction X, while the rear face 102R rests on the middle or ring fingers, and the thumbs rest on the front face 102F. Thus, when handling the device with both hands, the index finger 302L is naturally positioned on the sensors assembly 105, as shown in FIG. 7. In particular, the pulp 702 of the index 302L is naturally placed on the optical unit 116L to enable good quality optical measurement, while ensuring that a significant part of the contact surface 402 of the electrode 110L is in contact with the finger.

### Movable Optical Unit Adjacent to the Electrode

As may be seen from the figures, the optical unit 116L is adjacent to the contact surface 402 of the electrode 110L. More specifically, the interaction surface 604 of the optical unit 116L is adjacent to the contact surface 402 of the electrode 110L. By adjacent, it is meant that no mechanical parts are arranged between the contact surface 402 and the optical unit 116L (in a plane at the level of the contact surface 402). The contact surface 402 may be separated from the optical unit 116L by a gap of less than 1 mm, in particular less than 0.1 mm. Alternatively, the contact surface 402 is in direct contact with the optical unit 116L. The term “adjacent” underscores the proximity and absence of intervening components, enabling a seamless interaction between the two surfaces for accurate sensor measurements. The optical unit 116L may be designed to extend or retract slightly during operation, ensuring the gap remains minimal or non-existent, which enhances measurement consistency and user comfort. Furthermore, this configuration ensures that both the optical and electrical functionalities operate within the same physical zone, reducing the risk of misalignment and improving ergonomic integration. The interaction surface 604 is constructed to align closely with the user's contact point, optimizing signal acquisition while maintaining the compactness of the assembly.

This arrangement results in a very compact sensor, which interacts with the same area of the finger and maximizes contact between the sensor and the finger.

### Dome Structure

With reference to FIG. 9, at least part of the dome 902 is truncated cone-shaped, for example cylindrical. Its base is circular in the figures. The interaction surface 604 is then a disk and the lateral surface 606 is a cylinder. Alternatively, the base may be square or rectangular to form a parallelepiped.

The dome 902 may comprise a plastic material. In particular, the dome may be made entirely of plastic. Alternatively, the dome may comprise metal and/or glass.

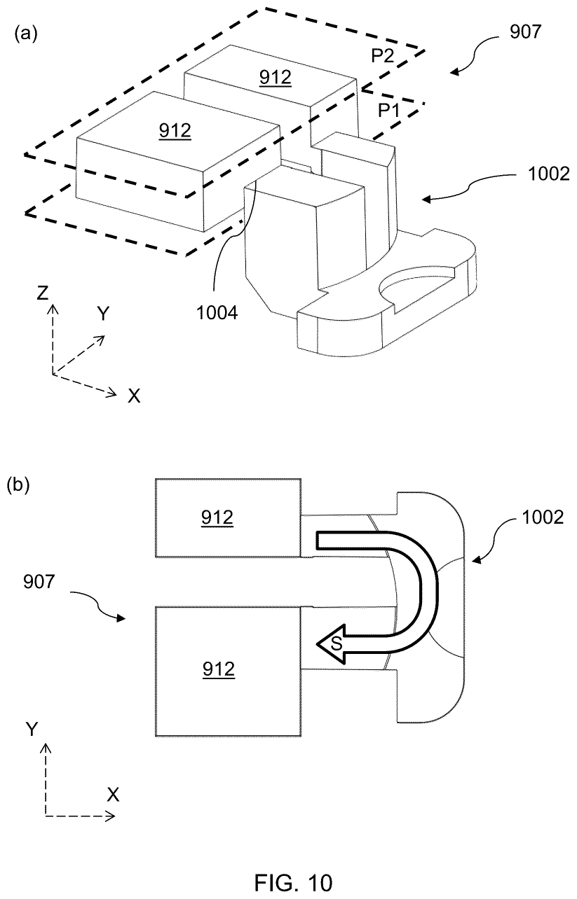

In an embodiment, the dome 902 comprises an opaque piece 905 and a transparent piece 907. The transparent piece 907 is shown in isolation in FIG. 10. The transparent piece 907 is configured to allow visible light to pass at least partially through it. This transparency means that the material of the transparent piece 907 allows the transmission of light within a specific wavelength range, enabling optical interactions, such as light emission and detection, relevant for the device's functionality. The degree of transparency can vary depending on the application, for example, the piece may be fully clear, transmitting nearly all visible light without distortion, or it may be semi-transparent, allowing some diffusion or attenuation of light for particular optical effects or measurement needs. In contrast, the opaque piece 905 is designed to block the passage of visible light or to substantially block the passage of visible light. By “substantially block the passage of visible light” it is meant than less than 10% of visible light is transmitted through the opaque piece 905. This light-blocking characteristic may be desirable to prevent interference from ambient light or to isolate the optical components housed within the dome. The material of the opaque piece 905 ensures that light is absorbed or reflected, thereby maintaining the integrity of the optical paths defined by the transparent piece. Together, the transparent and opaque components of the dome 902 create a controlled environment that facilitates precise light-based measurements while minimizing unwanted light interactions from external sources.

The opaque piece 905 may form the base 909, the side surface 606 and part of the interaction surface 604. The dome 902, and in particular the base 909, may be mounted on the support 504. In this way, the opaque piece 905 directly transmits the force exerted by the finger on the interaction surface 604 to the support 504.

Each piece 905, 907 is one-piece. In other words, each piece 905, 907 is formed from a single block of material. In particular, each piece is formed from a single piece of plastic material. The two pieces 905, 907 are molded together, in particular by bi-injection. Bi-injection enables the dome 902 to be produced with two successive injections of plastic. In particular, the process for producing the dome 902 comprises injecting a transparent plastic material into a first mold to form the transparent piece 907. The transparent part is then placed in a second mold and an opaque plastic material is injected into the second mold around the transparent part to form the opaque part. Alternatively, the process may be carried out with a single mold. The transparent part then remains in the mold and the second injection is made in this mold. The opaque material is molded over the transparent part to form the dome 902. The bi-injection process imparts structural features and functional benefits that are not achievable using other manufacturing methods. This technique ensures a seamless bond between the transparent and opaque parts, eliminating the need for adhesives or mechanical fasteners that could compromise the dome's optical or structural integrity. Moreover, bi-injection allows for precise alignment of the transparent and opaque components, ensuring optimal light transmission and blocking where required. The method also enhances the durability and reliability of the assembly, as the materials are fused during molding, creating a unified structure capable of withstanding mechanical stresses and environmental conditions. Additionally, bi-injection enables intricate designs and fine detailing, such as variations in thickness or specific transitions between the materials, which contribute to the overall functionality and aesthetic of the dome. These features make bi-injection an appropriate technique for manufacturing high-performance components like the dome 902.

The dome 902 comprises at the interaction surface 604 a respective transparent part 912 designed to be arranged opposite each light source 908 and each light receiver 910: in the figures, there are thus two transparent parts 912. In this way, the two transparent parts 912 are aligned in a direction orthogonal to the direction in which the contact surface 402 extends. As may be seen from the figures, the interaction surface 604 is formed by part of the opaque piece 905 and the plurality of transparent parts 912.

As shown in FIG. 9, the light source 908 is configured to emit visible light out of the dome 902 through the associated transparent part 912, and the light receiver 910 is configured to receive visible light from outside the dome 902 through the associated transparent part 912.

As shown in FIG. 10, the transparent parts 912 are connected by at least one connecting piece 1002. The transparent piece 907 thus comprises the plurality of transparent parts 912 and the at least one connecting part or piece 1002. In an embodiment, the transparent piece 907 comprises a plurality of transparent parts 912 and a connecting part or piece 1002 connecting the plurality of transparent parts 912.

As shown in FIG. 9, the transparent parts 912 may be placed at a distance from the optical sensor 906. In other words, the optical sensor 906 is not in contact with the transparent parts 912.

As shown in FIGS. 9 and 10, the thickness of the transparent parts 912 along the Z axis define a lower plane P1 and an upper plane P2. Each transparent part 912 may have a parallelepiped shape. Alternatively, each transparent part 912 may have a cylindrical shape.

Referring to view (b) of FIG. 10, the connecting piece 1002 extends between the two transparent parts 912 and forms part of a secondary optical path S, called an optical labyrinth, between the light source 908 and the light receiver 910. The optical labyrinth S is different from the main optical path P.

The connecting piece 1002 is designed so that the luminous flux passing through the optical labyrinth S is much lower, in particular at least ten times lower, than the luminous flux passing through the main optical path P. Thus, the luminous flux passing through the optical labyrinth S may be considered negligible compared to the luminous flux passing through the main optical path P. Consequently, the luminous flux picked up by the light receiver 910 may be considered to be essentially the flux coming from the main optical path P having passed through the user's body. In this way, the optical labyrinth S does not interfere with physiological measurements made with the optical sensor 906. This design is relevant for maintaining the accuracy of physiological measurements performed by the optical sensor 906. In an embodiment, by minimizing the influence of stray light or unwanted optical paths, the connecting piece 1002 ensures that the light captured by the light receiver 910 originates almost exclusively from the main optical path P after interacting with the user's body. This precise isolation of the desired optical signals eliminates potential noise or interference that could compromise measurement reliability. The connecting piece 1002 not only serves as a physical bridge between the transparent parts of the dome but also acts as a deliberate component in the optical design. It introduces directional changes and cross-sectional variations in the optical labyrinth S, further diffusing or scattering stray light. This mechanism ensures that the light signals relevant to physiological data are preserved with minimal distortion. By maintaining the fidelity of the main optical path, it enhances the sensitivity and accuracy of the device, enabling it to perform complex physiological measurements with high reliability. Without the carefully engineered features of the connecting piece 1002, the optical unit would be more susceptible to interference, leading to less precise data and reduced performance of the device. This makes the connecting piece a relevant element in the optical unit's design, safeguarding the integrity of its functionality.

To this end, as may be seen in view (a) of FIG. 10, the connecting piece 1002 extends over at least a portion entirely outside the zone delimited by the lower plane P1 and the upper plane P2 (i.e. the zone which is between the lower plane P1 and the upper plane P2) so that the labyrinth S extends, over a portion, entirely outside this zone. In other words, at least a part of the connecting piece 1002 is not located between the lower plane P1 and the upper plane P2. In particular, the transparent piece 907 extends at least in part in a direction that is not parallel to the interaction surface 604. In this way, light travelling along the secondary optical path S encounters several changes of direction, which greatly limits the flux passing through the secondary optical path S. The transparent piece 907 may comprise cross-sectional variations along the general direction of light flow, so as to further limit the luminous flux in the secondary optical path S. In particular, the transition 1004 between each transparent part 912 and the connecting part 1002 forms a significant cross-sectional narrowing (i.e. between the cross-section of the transparent piece 912 and the cross-section of the connecting part 1002). The connecting piece 1002 incorporates additional design elements to enhance its functionality. For example, the transparent piece 907 may include variations in cross-sectional shape along the general direction of light flow. These variations act as optical barriers or constrictions that further limit the passage of light through the secondary optical path S. The transition 1004, located between each transparent part 912 and the connecting piece 1002, is an example, featuring a significant narrowing in cross-section. This narrowing minimizes the amount of light that can bypass the intended path, adding an extra layer of control over stray luminous flux. The transparent piece 907 may also open onto the side surface 606. This design not only simplifies the manufacturing process but also aids in managing light distribution within the dome. The opening to the side surface can direct or diffuse light in specific directions, further reducing any interference that could affect the primary measurements. By incorporating these design features, the connecting piece 1002 serves as a relevant component in the optical unit, ensuring precision and reliability in its operation. Its careful placement and structural details optimize the interaction between light, the optical sensor, and the user's body, achieving a high standard of performance.

The transparent piece 907 thus enables easy dome production while greatly limiting bypassing of the secondary optical path S. Indeed, producing the dome 902 with a single transparent piece 907, and not with two different parts facing the source and receiver respectively, enables a faster and simpler manufacturing process to be used. In particular, with two separate transparent parts, it would be difficult to form the dome by bi-injection.

The optical unit 116L further comprises a cover 914 arranged in the inner volume 904. The cover 914 is arranged between the light source 908 and the light receiver 910, so that light cannot flow directly from the light source 908 to the light receiver 910 in the inner volume 904. The cover 914 divides the inner volume 904 into two distinct spaces. The light source 908 and the light receiver 910 are each arranged in a respective space.

The cover 914 may be made of a compressible material. For example, the cover 914 is made of elastomer. The cover 914 may be crushed during assembly between the dome 902 and the optical sensor 906 to ensure that no gap exists between the two spaces.

### Mobile Optical Unit

With reference to FIG. 7, the optical unit 116L may be movable relative to the contact surface 402 of the electrode 110L. In particular, the optical unit 116L is translationally movable along a direction orthogonal to interaction surface 604 (the Z direction in the figures, given that interaction surface 604 extends essentially along the X and Z directions). The optical unit 116L may be movable over a range of between 0.01 and 1 mm.

The optical unit 116L is movable between a rest position, shown in view (a) of FIG. 7, and a displaced position, shown in view (a) of FIG. 7. And conversely, the optical unit 116L is movable between the displaced position and the rest position. The rest position is the position of the optical unit 116L when the user is not exerting any force on the optical unit 116L, in particular when the user is not in contact with the optical unit 116L. The displaced position is the position of the optical unit 116L when the user exerts a force F on the optical unit 116L.

As shown in FIGS. 6 and 7, at least part of the optical unit 116L protrudes from the contact surface 402 in the rest position. In particular, the interaction surface 604 protrudes from the contact surface 402 in the rest position. Thus, at least part of the optical unit 116L extends beyond the extension of the contact surface 402. In the rest position, the user may therefore feel the asperity created by this projection under his finger. The optical unit 116L may protrude by a length of between 0.1 mm and 2 mm.

It is recalled here that the optical unit 116L is adjacent to the contact surface 402 of the electrode 110L. More specifically, the interaction surface 604 of the optical unit 116L is adjacent to the contact surface 402 of the electrode 110L.

### The Support

As may be seen in FIGS. 5 and 6, the dome 902 is mounted on the support 504, so as to close off the inner volume 904 of the dome 902. In this way, the dome 902, which comprises the interaction surface 604 on which the user presses, transmits the force of the finger directly to the support 504.

The support 504 comprises a front face 608 and a rear face 610. The optical unit 116L is mounted on the front face 608 of the support 504.

As explained above, the support 504 may be a printed circuit board, referred as optical printed circuit board, on which the optical unit 116L is mounted. The optical printed circuit 504 may be made of a metallic trace layer, e.g. copper, bonded to a dielectric layer, e.g. polyimide. The device 100 may comprise a main printed circuit board 802. In particular, the display 112 and the physical interface 114 are connected to and controlled by the main printed circuit board 802. The optical printed circuit board 504 is electrically connected to the main printed circuit board 802.

In an embodiment and with reference to FIG. 7, the support 504 is deformable. In particular, the support 504 is deformable by the force of a user's finger exerted on the optical unit 116L when it is movable relative to the electrode 110L. More precisely, the support 504 is deformable by the optical unit 116L (notably via the dome 902), which itself receives the force of the user's finger. The term “deformable” implies that the support is not rigid but is designed to have controlled elasticity or flexibility. This property is relevant for several reasons: it enables precise force measurements through the associated sensors, such as strain gauges or piezoelectric elements, and it prevents excessive stress on the components mounted on the support, thereby enhancing their durability. The degree of deformability is carefully calibrated to ensure that the support bends within a predefined range under typical user-applied forces. This range of deformation is sufficient to enable accurate measurement while avoiding permanent deformation or failure of the material. Materials used for the support, such as specialized plastics, elastomers, or composite materials, are selected for their ability to combine flexibility with strength and resilience. Additionally, the deformable nature of the support contributes to the ergonomic design of the device. It allows for a natural and intuitive user interaction, as the device responds to touch with subtle mechanical feedback. This feature ensures that the user's finger maintains consistent contact with the optical unit 116L and the electrode 110L, improving the accuracy and reliability of physiological measurements.

As mentioned previously, the support 504 is attached to the housing 102. In order to be deformed, In an embodiment, the support 504 is attached to the housing 201, in particular at two opposite ends 504a, 504b (for example opposite along the extension direction X, as illustrated in the figures, insofar as the support 504 has an elongated shape along this extension direction X). As may be seen in view (b) of FIG. 5, the support 504 is attached to the housing 102 at each of its ends, for example by a clamp 506. These two clamps 506 make the ends 504a, 504b of the support 504 fixed in the reference frame of the housing 102, so that the support 504 deforms between the two fixed ends, as shown in view (b) of FIG. 7. The clamps 506 hold the support 504 in the direction of movement of the optical unit, while simplifying assembly of the support in the housing.

### The Force Sensor

As may be seen from the embodiment shown in FIGS. 6 and 7, the force sensor 502 is a deformation sensor of the support 504. In this respect, the deformation sensor may be mounted on the support 504, for example on the rear face 610 of the support 504. In particular, the force sensor 502 is mounted on the side opposite the optical unit 116L. The force sensor 502 may be mounted substantially opposite the optical unit 116L. The term “substantially” in this context implies that the force sensor 502 is positioned in a way that is almost directly aligned with the optical unit 116L, but not necessarily perfectly so. This allows for minor deviations in placement without compromising the functionality of the sensor or the accuracy of its measurements. For example, the sensor could be slightly offset due to design constraints or material considerations while still effectively capturing the forces transmitted through the support 504. Alternatively, however, the force sensor 502 may be off-center on the support relative to the optical unit 116L.

The force sensor 502 may be a deformation sensor of the deformable support 504. The force sensor 502 is, for example, a piezoelectric deformation sensor. The force sensor 502 is configured to measure the deformation of the support 504. In particular, the force sensor 502 is configured to produce a signal for determining information relating to the force exerted on the optical unit 116L in the displaced position.

To this end, the device 100 comprises a force module 806 configured to receive signals from the force sensor 502 and to deduce information relating to the force exerted on the optical unit 116L. Typically, the force module 806 comprises an analog-to-digital converter (ADC) and a processor. The force module 806 may be mounted on the main printed circuit board 802 or on the optical printed circuit board 504.

Integrating the force sensor 502 directly onto the deformable optical printed circuit board 504 results in a simple, compact mechanical and electronic architecture. In fact, the optical printed circuit board 504 serves both as a support for the optical unit and as a strain gauge, in addition to supporting the printed circuit boards, thus limiting the problems of size, complexity, assembly and cost. This integration also eliminates the need for force propagation media and minimizes the number of parts, thus improving measurement of the force exerted by the finger.

In an embodiment, the device 100 further comprises a pressure module 808 configured to receive data from the optical module 612 and the force module 806 and to deduce a blood pressure of the user, in particular on the basis of the analysis of the pulsatility of the finger pressing on the optical unit with a pressure determined by the force module. Typically, the pressure module 808 comprises an analog-to-digital converter (ADC) and a processor. The pressure module 808 may be mounted on the main printed circuit board 802 or on the optical printed circuit board 504.

### User Feedback

In an embodiment, the device 100 is configured to provide the user with information representative of the signals received by the force module 806.

In particular, the display 112 may be configured to show information representative of the signals received by the force module 806. For example, the screen shows a recommended force interval to be exerted by the user on the optical unit 116L, notably to improve the quality of optical measurements.

Alternatively, or additionally, the device 100 may include a microphone. The microphone is then configured to output information representative of the signals received by the force module 806.

Alternatively, or additionally, the device may include a vibrator. The vibrator is then configured to provide haptic feedback information representative of the signals received by the force module 806.

### The Second Electrode

In an embodiment not shown, the device 100 includes a second sensors assembly so that in the two-handed handling position shown in FIG. 3, both sensor assemblies are positioned under a user's finger. The second sensors assembly may be similar to the sensors assembly 105 described above. Alternatively, the second sensors assembly may be different and, for example, comprise a single sensor.

In the alternative shown in the figures, the device includes a second electrode 110R positioned near the second end 103R so that in the two-handed handling position shown in FIG. 3, each electrode 110L, 110R is positioned under a user's finger.

The structure of the second electrode 110R is similar to that of the first electrode 110L and will not be described again. However, the second electrode 110L may differ from the first electrode 110L, particularly in that it has no opening for an optical unit.

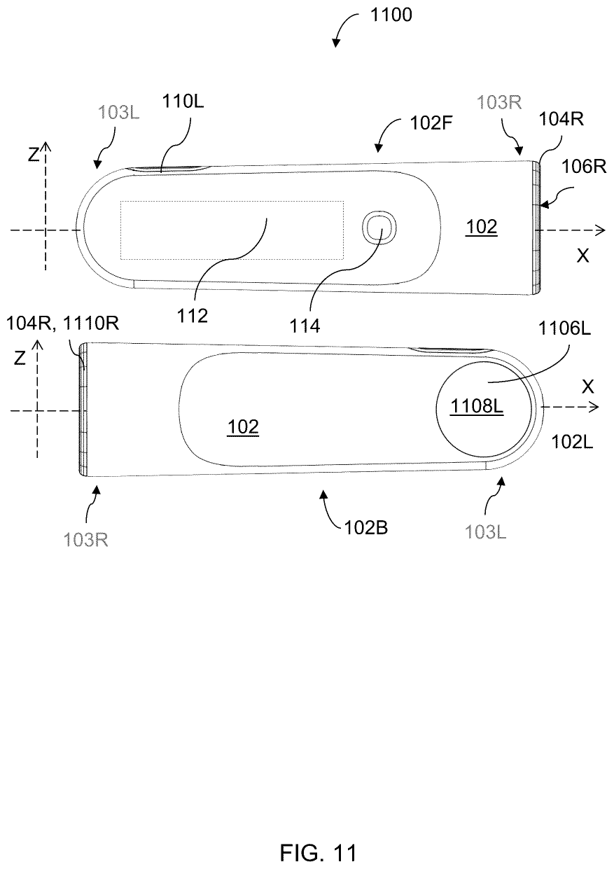

The second electrode 110L may be positioned in a similar way to the sensors assembly 105 as described above, i.e. both electrodes 110R, 110L are on the top face 102T (positioning symmetry visible on top face 102T in FIG. 2). More generally, the two electrodes 110L, 110R may be aligned parallel to the extension direction X. Symmetrical positioning simplifies measurement. Alternatively, the first ECG electrode 110L may be on the top face 102T and the second electrode 110R may be on the front face 102F or on the second end 103R, as shown in FIG. 11.

The second electrode 110L may be an ECG and/or IPG electrode. By simultaneously touching the two ECG electrodes with two fingers of different hands, the user may perform an ECG. Alternatively, by simultaneously touching the two IPG electrodes with two different fingers, the user may perform an impedance measurement.

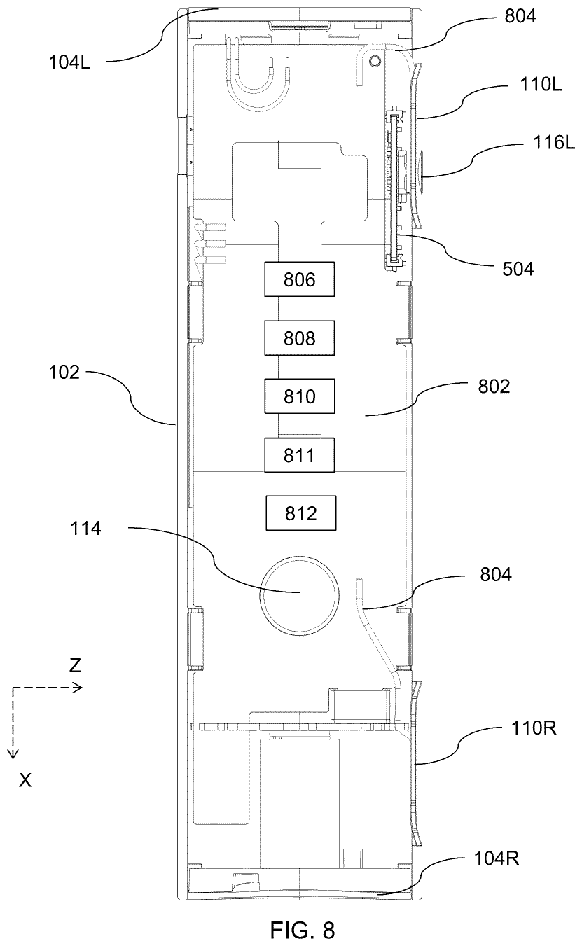

As shown in FIG. 8, the two electrodes 110L, 110R are connected to the main circuit board 802, in particular via the electrical contacts 804.

The device 100 may comprise an ECG module 810 connected to the two ECG electrodes 110L, 110R. Typically, the ECG module 810 comprises an analog-to-digital converter (ADC) and a processor. The ECG module 810 may be mounted on the main printed circuit board 802.

The ECG module 810 is configured to retrieve electrical signals from the human body via the ECG electrodes 110L, 110R and, after processing, to generate an electrocardiogram.

In an embodiment, the ECG module 810 is configured to impose a potential on one of the two ECG electrodes, and the potential at the other electrode is left free by the ECG module 810. In this way, the potential of this electrode corresponds to the potential of the user's body (when there is contact) and varies according to the user's heartbeat.

The device 100 may include an IPG module 811 connected to the two IPG electrodes 110L, 110R. Typically, the IPG module 811 comprises an analog-to-digital converter (ADC) and a processor. The IPG module 811 may be mounted on the main printed circuit board 802.

The IPG module 811 is configured to inject an electric current into the human body via the IPG electrodes 110L, 110R and, after processing, to determine, for example, a user's body composition.

In an embodiment, the device further comprises a wave module 812 configured to calculate a propagation velocity of a pulse wave in a user's arm based on signals received from the ECG module and the optical module. Typically, the wave module 812 comprises an analog-to-digital converter (ADC) and a processor. The Wave module 812 may be mounted on main printed circuit board 802 or on optical printed circuit board 504.

### Additional Physiological Sensors on the Extremities

In an embodiment, the device 100 further comprises an additional physiological sensor 106L at the first end 103L, referred to as the first additional physiological sensor 106L.

The additional first physiological sensor 106L comprises a functional surface 108L. By functional surface 108L, it is meant a surface intended to be positioned facing the user, to interact with the latter, with or without contact, to obtain the physiological measurement by the first physiological sensor 106L.

For example, the first additional physiological sensor 106L may be an electronic stethoscope, with a piezoelectric sensor and an amplification membrane designed to be positioned on the user. In this case, the functional surface 108L comprises the amplification membrane. In particular, the amplification membrane is the part of the piezoelectric sensor visible to the user. The membrane may typically be constructed from a durable, acoustically efficient material, such as polymer composites or high-grade elastomers, chosen for their ability to vibrate in response to sound waves while minimizing distortion. The thickness of the membrane may be within a range of 1 mm to 3 mm, notably 2 mm. A thinner membrane may enhance the sensitivity to faint sounds, whereas a slightly thicker membrane improves durability and resistance to wear during repeated use.

For example, the first additional physiological sensor 106L may be a thermometer, with a thermopile-type sensor and a lens. The lens may be surrounded by a cone. In this case, the functional surface 108L comprises the lens and, if applicable, the cone. In particular, the lens and cone are the parts of the thermometer that are visible to the user.

For example, the first additional physiological sensor 106L may be a spirometer with an air volume and/or flow sensor and a mouthpiece. In this case, the functional surface 108L comprises the mouthpiece. In particular, the mouthpiece is the part of the spirometer visible to the user.

The first additional physiological sensor 106L is positioned at the first end 103L and its functional surface 108L is inscribed within the edge 104L (i.e. the functional surface 108L is integrated within the boundary of the edge 104L). This means that, in a projection along the extension direction X in a transverse plane YZ at the end 103L, the functional surface 108L is positioned inside the edge 104L. Formulated differently, the projection of functional surface 108L is included in the projection of the side face. In other words, the projection of the functional surface 108L lies entirely within the outline of the side face defined by the edge 106L.

Thanks to these features, holding the device 100 is not hindered by the physiological sensor 106L. In particular, the user may comfortably hold or grip the device 100 by one end, with the hand (e.g. palm) aligned along the direction of extension. This feature enhances the device's practicality by making it compact and easy to store in a pocket, box or similar space.

In this respect, in an embodiment, the functional surface 108L is positioned within the volume defined by the housing 102 (and therefore by the edge 104L at the end 103L). The sensor 106L therefore does not protrude from the housing 102. However, for some sensors, particularly those requiring contact, such as the stethoscope, the functional surface 108L (e.g. the membrane) may protrude a maximum of 5 mm from the volume defined by the housing 102 along the main direction X, or even a maximum of 2 mm.

In an embodiment, the device 100 comprises an additional second physiological sensor 106R at the second end 103R. This second physiological sensor 106R is defined similarly to the first additional physiological sensor 106L.

In an embodiment, illustrated in FIG. 1, the first additional physiological sensor 106L is an electronic stethoscope and the second additional physiological sensor 106R is a temperature sensor.

Alternatively, the first additional physiological sensor 106L is a temperature sensor and the second additional physiological sensor 106R is an electronic stethoscope.

### Device Variant

FIGS. 11 and 12 illustrate another embodiment of a device 1100. This embodiment is similar to the device 100, except that a physiological sensor 106L, 106R is not inscribed in an edge as previously described.

In FIG. 11, the first physiological sensor 1106L is modified, but it could be the second physiological sensor 1106R.

In this embodiment, a physiological sensor 1106L comprises a functional face 1108L that is positioned on the front face 102F or the rear face 102R. In the illustrated example, the functional surface 1108L is positioned on the rear face 102R, on the opposite side to the display 112 and the mechanical interface 114. In particular, sensor 1106L is a stethoscope and functional interface 1108L is a membrane.

However, the physiological sensor 1106L remains near to the end 103L, with “near” meaning within half or a quarter of the length L of the device 1000.

The user may hold the housing 102 between the physical interface 114 and the second edge 104R to apply the membrane 1108L to the torso.

As previously described, FIG. 12 illustrates a further embodiment of the second electrode 1100R. This is not directly related to the stethoscope described above.

Indeed, the second electrode 1100R may be positioned on all or part of the edge 104R of the end 103R. In this way, contact is no longer made by the finger but by the palm of the hand, in a two-handed handling position.

The sensor may be made by depositing metal on the edge or by adding a metal part.

As previously explained, the contact surface 402 here has a concave shape. In other words, the contact surface 402 has a domed shape towards the inside of the housing 102. This embodiment is not directly related to the stethoscope and second electrode described above.

### The Device and its Environment

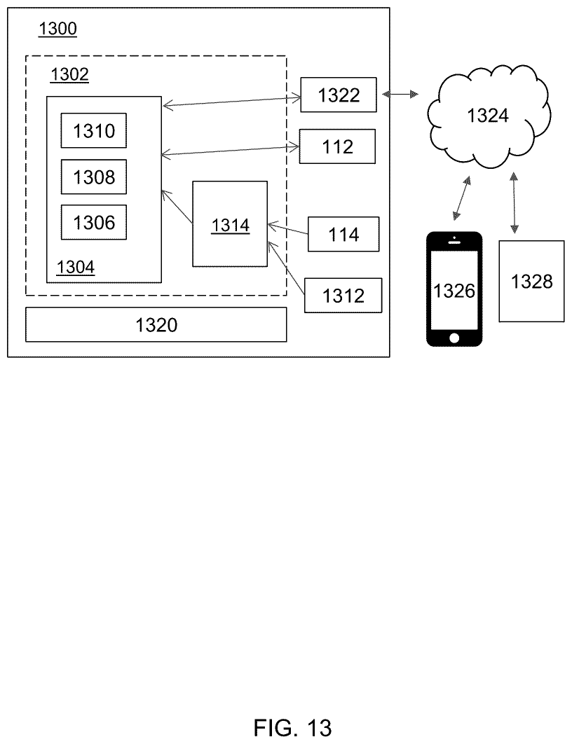

FIG. 13 shows a schematic diagram of the architecture of a device 100, 1300 (referenced 1300 on this figure) as described and its environment.

The device 1300 comprises a control unit 1302 with control circuitry 1304 including a processor 1306, memory 1008 and an I/O interface 1310 (“In/Out”) for communicating with other components.

The memory 1308 stores programs, instructions or other items that enable navigation on the device 1300 and measurements to be taken (algorithms in particular). In particular, the memory 1308 breaks down into a volatile memory, of the RAM type, and a non-volatile memory, of the flash (or ROM or SSD) type.

The control unit 1302 is configured to control the ECG module, the optical module, the force module, the pressure module and/or the wave module. In particular, the control unit 1302 is configured to control the simultaneous measurement of an ECG, via the ECG module, and an optical measurement, via the optical module.

In particular, the control unit 1302 is arranged on the main printed circuit board 802. The control unit 1302 may consist of several sub-units, arranged on the main printed circuit board 802 and on the optical circuit 504. The device 1300 comprises one or more sensors 1312 (all the sensors described above are shown under a single reference 1312).

The control unit 1302 typically comprises an interface module 1314 interfacing between the sensors 1312 and the I/O interface 1310 of the control circuitry 1304. The interface module 1314 includes ADCs, filters, amplifiers, etc.

The device 1300 further comprises the display 112, which communicates with the I/O interface 1310, and the physical interface 114, which communicates with the interface module 1314 for navigating the display 112 menu.

To supply the various components with electrical power, the device 1300 comprises a battery 1320, for example a rechargeable battery. The battery 1320 is configured to power the control unit 1302, the display 112 and the sensors 1312.

Finally, for connectivity, the device 1300 comprises a wireless communication module 1322 (BLUETOOTH® (a short-range wireless technology standard), BLE (BLUETOOTH® low energy), Wifi, cellular, etc.), connected to control circuitry 1302. The module communicates 1322 via a communication network 1324 with a mobile terminal 1326 (e.g. a smartphone-type cell phone) and/or a remote server 1328. The physiological data thus acquired by the device 1300 may be stored, analyzed, processed in the server 1328 and displayed by the mobile terminal 1326. The mobile terminal 1326 may also act as a relay between the device 1300 and the server 1328 (e.g. in the case of BLUETOOTH® or BLE communication).

Expressions such as “comprise”, “include”, “incorporate”, “contain”, “is” and “have” are to be construed in a non-exclusive manner when interpreting the description and its associated claims, namely construed to allow for other items or components which are not explicitly defined also to be present. Reference to the singular is also to be construed in be a reference to the plural and vice versa.

The articles “a” and “an” may be employed in connection with various elements and components, processes or structures described herein. This is merely for convenience and to give a general sense of the compositions, processes or structures. Such a description includes “one or at least one” of the elements or components. Moreover, as used herein, the singular articles also include a description of a plurality of elements or components, unless it is apparent from a specific context that the plural is excluded.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

A person skilled in the art will readily appreciate that various features, elements, parameters disclosed in the description may be modified and that various embodiments disclosed may be combined without departing from the scope of the invention. For example, various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Having described above several aspects of at least one embodiment, it is to be appreciated various alterations, modifications, and improvements will readily occur to those skilled in the art. Such alterations, modifications, and improvements are intended to be aspects of this disclosure. Accordingly, the foregoing description and drawings are by way of example only.

1. A sensors assembly for physiological measurements comprising:
an electrode comprising a contact surface, the contact surface being configured to be in contact with a user, the contact surface being elongated along a main direction,
an optical unit comprising an optical sensor and an interaction surface configured to be in contact with a user,
wherein the contact surface comprises a first section and a second section along the main direction,
wherein the interaction surface is positioned along the main direction between the first section and the second section,
and wherein the first section has a greater length than the second section along the main direction.

2. The sensors assembly of claim 1, wherein the contact surface defines an aperture, the interaction surface being positioned in the aperture.

3. The sensors assembly according to claim 1, wherein the contact surface surrounds the interaction surface.

4. The sensors assembly according to claim 1, wherein the first section and the second section of the contact surface are separated from each other.

5. The sensors assembly according to claim 1, wherein the contact surface is adjacent to the interaction surface of the optical unit.

6. The sensors assembly according to claim 1, wherein the optical sensor comprises a light source and a light receiver, the light source and the light receiver being aligned in a direction orthogonal to the main direction in which the contact surface extends.

7. The sensors assembly according to claim 1, wherein the interaction surface has an area twice smaller than the area of the contact surface.

8. The sensor assembly according to claim 1, wherein the interaction surface has an area three times smaller than the area of the contact surface.

9. The sensors assembly according to claim 1, wherein the electrode is an ECG electrode.

10. A device comprising a housing and the sensors assembly according to claim 1, the sensors assembly being arranged on the housing.

11. The device according to claim 10, wherein the electrode is mounted on the housing and the contact surface has a concave shape relative to the housing so as to form a channel for receiving the finger.

12. The device according to claim 10, wherein the housing has an elongated shape along a direction of extension between two ends, the main direction of the contact surface being parallel to the direction of extension of the housing.

13. The device according to claim 10, wherein the contact surface is located near one end of the device along the direction of extension, the first section being located between the end and the second section along the direction of extension.

14. The device according to claim 10, wherein the contact surface near a distal end of the device along the extension direction is disposed between a first edge of the housing and strictly a quarter of a length of the housing along the extension direction from the first edge.

15. The device according to claim 10, wherein the device is holdable with two hands in use.

16. The device according to claim 10, wherein a section of the housing is oblong or rectangular, wherein the housing comprises a front face, a rear face, a top face, and a bottom face, opposite the top face, wherein the front and rear faces have a height greater than the depth of the bottom and top faces, wherein the sensors assembly is positioned on a top face of the housing.

17. A sensors assembly for physiological measurements comprising:
an electrode comprising a contact surface, the contact surface being configured to be in contact with a user, the contact surface being elongate along a direction of extension, the contact surface defining an aperture, the aperture being off-center from the contact surface along the direction of extension, and
an optical sensor arranged in the aperture.

18. The sensors assembly according to claim 17, wherein a barycenter of the contact surface and a barycenter of the aperture do not coincide.
